(19) United States
(12) Patent Application Publication
(10) Pub. No.: US 2008/0009951 A1
(43) Pub. Date: Jan. 10, 2008

(54) Femoral head resurfacing implant with internal plate fixation and instrumentation

(76) Inventor: W. Andrew Hodge, Boynton Beach, FL (US)

Correspondence Address: MCHALE & SLAVIN, P.A., 2855 PGA BLVD, PALM BEACH GARDENS, FL, 33410, US

(21) Appl. No.: 11/772,450

(22) Filed: Jul. 2, 2007

Related U.S. Application Data
(60) Provisional application No. 60/806,387, filed on Jun. 30, 2006.

(51) Int. Cl. A61F 2/38; A61B 17/58

(52) U.S. Cl. 623/20.35; 606/69

(57) ABSTRACT

The instantly disclosed implant is designed to resurface or partially replace (two versions) the arthritic or osteonecrotic femoral head in an anatomic fashion with maximum fixation, durability and stability. It will serve a wide range of patient ages, arthritic/traumatic deformity, and bone pathologies while providing for high performance activity. The implant has absolute fixation utilizing existing, contoured femoral head bone with or without bone cement/porous texturing, an internal plate with modular head attachment, and advantageously benefit from the concurrent use of precision guided instrumentation. This implant optimizes maximal femoral head contact with or without porous contact surfaces but does not rely purely on the head for long term stability. This new concept prevents the common failure mechanisms of femoral neck fracture, loosening and malpositioning as well as makes patient bone quality less important for this high performance hip resurfacing implant.

### REFERENCE TO RELATED APPLICATIONS

This application claims benefit of the filing date of U.S. Provisional Application No. 60/806,387, filed on Jun. 30, 2006, the contents of which are herein incorporated by reference.

### FIELD OF THE INVENTION

This invention relates to a femoral head resurfacing or partially replacing implant combining internal plating with other fixation and instrumentation systems.

### BACKGROUND OF THE INVENTION

Hip resurfacing surgery began in the 1920s, and since that time has undergone several design modifications. Between the 1920s to the 1940s, early attempts at resurfacing the femoral head, known as “mold arthroplasty” were practiced. Between 1950 through the 1980s, a second generation was introduced which included an acetabular component. Beginning in the 1990s, a third generation design was introduced, which is gaining popularity today. Studies of the clinical results and modes of failures in each of these technologies was discussed in an article by the present inventor in Seminars in Arthroplasty (17: 35-41, 2006), and are summarized herein.

The concept of hip resurfacing dates back over 80 years. The first generation consisted of the original mold arthroplasty design by Smith-Petersen in 1923, who reshaped the femoral head and covered it with a cup that at first was made of Pyrex glass. Unfortunate catastrophic cup failure resulted, causing him to switch to a biologically inert metal called VITALLIUM, which represented a “metal mold arthroplasty of the hip.” This mold arthroplasty of the femoral head was press-fit having varied fixation success and unpredictable results. The subsequent history of hemi-resurfacing of the hip followed with small modifications as seen with designs such as the adjusted cup, spherocylindric cup, and Thomine cup.

The concept of resurfacing the arthritic socket as well as the femoral head led to a second generation of hip resurfacing which began in the early 1950s. In 1951, Charnley's press-fit Teflon-bearing design was the first total hip resurfacing system. The poor wear characteristics of Teflon contributed to the early failure of these systems. Subsequently, the concept was also driven by the novel use of dental acrylic cement in the hip. Haboush, in 1953, first reported on the use of methacrylate cement in two cases of “double metal cup arthroplasty” or the first cemented “total resurfacing hip arthroplasty.” The designs continued to evolve through the 1960s and 1970s with metal-on-polyethylene designs and early metal-on-metal (MOM) designs. The designs of the 1970s and 1980s were characterized by metal-on-polyethylene articulation, fixed either with or without cement. In the uncemented category, the TARA design (Depuy, Warsaw, Ind.) was the first to re-move a portion of the proximal femoral head to facilitate support of the prosthesis. This design also employed a curved stem to facilitate positioning by cannulating the femoral neck. Failure of these designs was primarily related to instability of the device on the femoral head. These designs were replaced by cemented designs which reduced femoral instability to a low rate. Despite the advantage of cement, these early designs continued with a higher rate of revision due to femoral neck fractures, and continuing or exacerbation of pain. In 1967, Muller began using metal-on-metal resurfacing with some good results that would last 25 years. This early metal-on-metal hip resurfacing suffered from inconsistent manufacturing and therefore inconsistent results, which he abandoned in favor of the evolving standard of total hip replacement (THR).

In 1973 Eicher and Capello developed a cemented total hip resurfacing prosthesis with a metal femoral and polyethylene acetabular component. In 1975 Amstutz and coworkers introduced the THARIES system, which was also cemented metal-on-polyethylene. In the 1980s, Amstutz and colleagues evolved the design to incorporate cementless fixation. These designs were plagued by the wear properties of the materials chosen. The first-generation polyethylene had high wear rates, which were compounded by the large-diameter heads and thin liners. The resulting polyethylene debris created large areas of osteolysis behind the cup and in the proximal femur. At the time, however, osteolysis was not recognized as such and the failures were attributed to other factors such as avascular necrosis and high frictional torque forces on the acetabulum. In addition, these early designs were prone to femoral neck fracture. The osteolysis most likely contributed to this failure mode as well as intraoperative neck notching, which was a consequence of the recommended extreme valgus placement and undersizing of the implant to reduce frictional torque.

The third and current generation of hip resurfacing technologies began emerging in the early 1990s. These designs usually have large metal-on-metal articulations and use either cementless or hybrid fixation. The first design was the Wagner cementless resurfacing system, which utilized a wrought Co—Cr alloy with tightly controlled component tolerances to minimize wear. This early design was not widely adopted due to implantation difficulties. No long-term data on the survivability of this design are in print.

McMinn introduced a cementless resurfacing system in conjunction with Corin Medical (Cirencester, UK) in 1991. The initial press-fit design was plagued by component loosenings, which were addressed by adding a roughened surface and hydroxyapatite. A cemented version was then introduced that had a high incidence of cement-cup debonding. A hybrid system with a cemented femoral component and a cementless hydroxyapatite-coated cup was then introduced in 1994. The designs of Corin and McMinn then diverged in 1996 with the McMinn prosthesis evolving into the Birmingham Hip Resurfacing (Smith & Nephew, Memphis, Tenn.) and Corin's system becoming the Cormet 2000 Hip Resurfacing System. Also in 1996, Amstutz designed the Conserve Plus hybrid hip resurfacing (Wright Medical Technology, Arlington, Tex.). These three designs are the most prevalent of the third-generation devices that are in use today worldwide. All of these third-generation systems have the common features of metal-on-metal articulation using Co—Cr, cementless acetabular fixation, and cemented femoral fixation.

There has been a resurgence of interest in resurfaced hips because of the advent of metal to metal articulations and the application to high performance patients. Despite the new resurgence of resurfacing technology, the problems of variable bone quality and demanding surgical technique still exist, limiting the application of this technology.

Although the current hip resurfacing technology has largely solved the polyethylene osteolysis issue through metal articulations, patient selection and surgical technicalities have remained difficult. The inability to analyze poor bone pre-operatively is a decided drawback, given that the presence of this poor bone in the femoral head can cause early loosening or early femoral neck fracture. The current surgical technique is quite confusing with less than adequate guidance for the average surgeon.

If an implant could be developed suitable to fortify against the common failure modes of existing hip resurfacing implants, mainly early femoral neck fracture and femoral loosening, a long felt need would be satisfied.

### PRIOR ART

Bosacco, U.S. Pat. No. 3,670,724, discloses a hip replacement prosthesis comprising an artificial ball mounted on or integral with an intermediate portion which abuts the end of the bone in question. A shank or stem having a plurality of screw holes is attached to the intermediate portion and is intended for intramedullary insertion. The intermediate portion has at least one locating hole and the stem has a plurality of spaced holes formed therein. The stem is inserted into the medulla leaving the locating holes in the intermediate portion exposed. A rectangular post is fitted into the locating holes and then a template having a rectangular aperture is fitted over the rectangular post. The template has a plurality of screw holes in it positioned, when in place, to be congruent with the holes in the stem. When the template is in place one or more holes are drilled transversely through the template, the near bone cortex, the corresponding prosthetic stem hole and the far cortex portion. The template is then removed and screws are then screwed through the aligned holes in the bone and the stem. In ordinary bone fixation a similar technique is used except that at least part of the external plate or template is left on and the screws are screwed through it, the bone and the intramedullary nail.

Johnston, U.S. Pat. No. 3,765,034, discloses a hip joint prosthesis of generally conventional configuration in that it includes an elongated tapered spindle curving slightly laterally at its major dimension end with the latter including a partial spherical head for universal engagement in a pelvis mounted socket provided therefore. The prosthesis is alleged to depart from the conventional prosthesis or similar apparatus in that the spindle is provided with longitudinally spaced and transversely extending apertures and one side of the head is provided bores with laterally outwardly opening parallel bores. A combined jig and drill guide is also provided and includes a plurality of locating pins telescopingly receivable in the bores and sleeve portions supported in fixed relation relative to the locating pins aligned with the apertures when the guide has its locating pins disposed in the bores. The spindle is first driven into the medullary cavity and thereafter the guide is positioned alongside the femur with the locating pins received in the bores. Thereafter, a suitable drill may be inserted through and guided by the sleeve portions for drilling bores in the femur aligned with the apertures formed in the spindle disposed in the medullary cavity. After removal of the drill guide suitable threaded fasteners may be threaded through the bores formed in the femur and the apertures formed in the spindle of the prosthesis to thereby lock the spindle in fixed relation relative to the femur so as to eliminate relative movement between the prosthesis spindle and the femur.

Tepic, U.S. Pat. No. 5,458,654, discloses a femoral component for a hip joint prosthesis with an intramedullary stem having a free distal region, a proximal region extending to a neck for receiving a ball head, a medial side and a lateral side. The stem has screw holes extending from said lateral side to said medial side for receiving bone screws in the lateral to medial direction for fixation of said stem to the medial cortex. The stem is coupled primarily to the medial cortex of the femur and only secondarily to the cancellous portion of the proximal femur to aid the rotational (torsional) stability of the implant.

Townley, U.S. Pat. No. 6,096,084, discloses a modular ball and socket joint has 1) a cupped ball head, preferably of ceramic, having a support body with an inferior, deep, distally facing, preferably generally planar, surface having a substantially circular outer boundary thereto; a distally opening stem receiving bore preferably centrally located in the support body; a cup wall, extending distally from the support body and having a preferably substantially cylindrical inner surface which extends from said outer boundary of said distally facing surface; and a superficially facing, generally semispherical, smooth external surface, preferably and optimally of a low friction coefficient, encapsulating the support body and cup wall; and 2) an interchangeable and modular stem, preferably of metal or metal alloy, having a distally directed spike, and a superior stem cap which is insertable into the bore of the head; optionally with 3) a head-receiving articular cup having an inner articular surface and a mountable back surface, the articular surface of which, when the head-receiving cup is suitably mounted in suitable receiving stock, mates in articulating contact with said smooth external surface of said head when said head and stem are suitably mounted in suitable receiving stock. The head and stem modularity allows for noticeably increased versatility and efficiency in surgical procedures, and the ceramic material is beneficially adapted for employment in the fabrication of femoral resurfacing implants for conservative arthroplasty.

Tepic et al., U.S. Pat. No. 6,409,768, discloses a screw-based primary fixation of the prosthetic components within the medullary cavity solves the problem of micro-movements encountered in conventional press-fit cementless fixation. For a total hip prosthesis, the stem is fixed to the medial cortex of the proximal femur by the medial approach alone, obviating the need for drilling of the lateral cortex. The stem may be implanted using special drill guide instrumentation. Anchoring screws are locked into the stem of the femoral component, while self-cutting threads on the screw head engage the pre-drilled medial cortex. This fixation principle is suggested to be applicable to other joint prostheses, e.g. finger, shoulder, elbow and knee, as well as to dental and spinal implants.

Lakin, U.S. Publication No. 2006/0241779, discloses femoral head modular resurfacing systems. The systems primarily include a head component and a stem component. The configuration of the head component and stem components allow for minimum invasiveness into the femur head region, thus conserving greater amounts of bone tissue than would be possible with conventional hip replacement systems. The systems also provide for various angles and offsets to be achieved between the systems and the femur head. The systems are useful in partial hip replacement procedures, as well as total hip replacement procedures, in which case an optional acetabular component would also be employed.

Storer, et al U.S. Pat. No. 6,524,343. is directed toward a prosthetic femoral component located in a prepared socket in a femur which has been resected at a position on the proximal side of its neck and includes a tapered insert and a proximal head portion. The proximal end of the insert portion is adapted for location in the prepared socket and has a maximum dimension in a plane normal to the distal-proximal axis of the neck which is larger than the minimum dimension of the neck in a parallel plane. The component takes advantage of the bone at the periphery of the socket which enables the insert to be accurately and firmly located in the bone. The presence of the bone at the outer edges of the socket helps to stabilize the component. In one embodiment, a tapered insert portion is flared outwardly in the proximal direction, and can be dimensioned to pass through the neck of the femur with which it is to be used or it can stop short of it.

The prior art references only suggest implantation of a femoral resurfacing prosthesis into cancellous bone. Such a prosthesis is prone to failure since the cancellous pocket is incapable of supporting the proximal femoral loads, which are generally on the order of 5-6 times body weight. As opposed to the present invention, the prior art fails to teach or suggest a femoral resurfacing prosthesis which positively fixates into cortical bone. Additionally, the present invention differs in teaching embodiments which eliminate rotation of the femoral ball or partial replacement, thereby providing enhanced long-term stability.

### SUMMARY OF THE INVENTION

The instantly disclosed implant is designed to resurface or partially replace (two versions) the arthritic or osteonecrotic femoral head in an anatomic fashion with maximum fixation, durability and stability articulating with a normal or replaced acetabular socket. It will serve a wide range of patient ages, arthritic deformity, and bone quality and provide for high performance activity. The instantly disclosed implant may be of unitary or modular design, and will be useful where varying degrees of head arthritis, deformity, or necrosis are present.

The present device resurfaces or partially replaces the arthritic femoral head while insuring absolute long term fixation in the face of a variety of pathologic bone deformities and qualities. All embodiments of the implant provide absolute fixation utilizing existing, contoured femoral head bone with or without bone cement, and an internal intramedullary plating system with integral or modular head design. The implant optimizes maximal femoral head contact but does not rely purely on the head for long term stability. This new concept prevents the common failure mechanisms of femoral neck fracture, loosening and malpositioning as well as simplifying patient selection and broadening application by making absolute bone quality less important for this high performance hip resurfacing implant. Additionally, the implant may contain means to further provide anti-rotation of the head, whether in a modular or unitary configuration.

In one embodiment, the implant has a modular connection between the articular head surface and an internal plating (stem) system for optimal flexibility and ease of implantation and inventory consideration. The optimal strong bone of the medial femoral calcar is used for screw fixation of the internal femoral plate (stem) which stabilizes the resurfacing or hemihead replacement head and prevents femoral neck fractures or loosening. The implant's minimally invasive nature provides maximum bone conservation, wherein the use of screws guarantees long term fixation, thereby solving a long-standing problem with existing resurfacing implants. This large femoral head implant will articulate with a variety of acetabular implant component configurations and materials, as well as normal sockets.

In a minimally invasive way, this device resurfaces or partially replaces the damaged femoral head in a wide variety of pathological conditions and deformities especially in the young, high performance population. This implant will give the surgeon maximum flexibility to do a resurfacing or partial replacing type hip implant more reliably and in a wider variety of conditions and age groups than currently possible with existing technology. The surgeon will be able to implant the hip in a much safer, simplified, longer lasting and stronger fashion than has heretofore been possible with existing implants. The implant will have optimal hip joint anti-dislocation stability because of the large head and anatomic shape. Optimal fixation is provided due to the modular or integral internal intramedullary plating (stem) system, and the use of precision minimally invasive guided instrumentation.

In a preferred, albeit non-limiting embodiment, the internal plating component is constructed and arranged to enable deformation of the stem upon fixation to the dense calcar. This deformation causes the stem to closely approximate the natural configuration of the calcar, and thereby provide dynamic bone loading via the screw fixation. This is theorized to enhance bone growth, and encourage long-term preservation of the calcar. Furthermore, via the optional inclusion of an anti-rotation construction, undesirable rotation of the resurfacing or hemihead replacement head may be realized. With this internal strutting of the femoral neck, femoral neck fractures can be greatly reduced or avoided.

Accordingly, it is an objective of the instant invention to provide an internal plate fixation system on the strong medial femoral calcar. The internal plate system assures absolute instant fixation in a wide variety of hip deformities and bone qualities which have been previously very problematic.

It is a further objective of the instant invention to provide a system which may advantageously utilize unique, less invasive, precision guided instrumentation. This instrumentation allows easy identification of the center of the femoral neck and after seating of the implant inside the neck, uses precision guides for external drilling through the dense calcar bone linking to the implant holes. After accurately securing the internal plate, it becomes simple to ream the femoral head correcting any pathologic malposition to a more normal anatomy.

It is yet another objective of the instant invention to provide an embodiment utilizing modularity which makes implant fitting easier.

It is a further objective of the instant invention to provide an embodiment which combines a unitary construction having a shaped internal plating component coupled to alternative head construction designs. This enables absolute fixation while simultaneously providing the surgeon with a variety of bone conserving alternatives.

It is a still further objective of the instant invention to provide a resurfacing or hemihead replacement head which engages the prepared damaged femoral head and/or the internal plating component to prevent rotation.

It is a yet a further objective of the invention to provide an ability to replace or stabilize poor or missing femoral head bone stock.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

These and other advantages and features of the invention will become apparent upon reading the following detailed description and referring to the accompanying drawings in which like numbers refer to like parts throughout and in which:

FIGS. 1A and 1B show a metal-on-metal resurfacing implant typical of the prior art;

FIG. 2 is directed toward a prior art total hip prosthesis which incorporates screw fixation to the medial cortex;

FIG. 3 is a side perspective view of a modular femoral head resurfacing implant of the invention, as positioned upon a resurfaced femoral head, inclusive of an internal plating component affixed to the medial femoral calcar;

FIG. 4 is a cross-sectional view of a modular femoral head resurfacing implant, shown in anti-rotational engagement with both a resurfaced femoral head and an internal plating component which is in turn affixed to the medial femoral calcar;

FIG. 5 illustrates preparation of the femoral head and neck prior to insertion and fixation of the internal plating component of the modular embodiment of FIG. 3;

FIG. 6 illustrates insertion and fixation of the internal plating component of the modular embodiment of FIG. 3;

FIG. 7 is a cross-sectional view along line 6-6 of FIG. 6, which illustrates the mechanical engagement of the drilling guide and internal plating component for alignment of the holes drilled through the medial femoral calcar;

FIG. 8 represents a cross-sectional view illustrating preparation of the femoral head and neck for accepting the prosthesis components;

FIG. 9 shows an internal; plate for insertion within the prepared femoral head and neck of FIG. 8;

FIG. 10 illustrates a perspective view of the resurface femoral head of FIG. 7, into which the internal plating system has been positioned and affixed, and shown engaged with the drilling guide;

FIG. 11 shows a prosthetic femoral head of the present invention a illustrating bone compartment, anti-rotation wall and taper;

FIG. 12 shows an alternative configuration for an anatomically compliant internal plating component, useful in either a modular or a unitary femoral resurfacing or hemihead implant design;

FIG. 13 shows a resurfacing prosthesis in accordance with FIG. 12, illustrating mechanical cooperation with a drill guide;

FIG. 14 illustrates an alternative intramedullary stem construction;

FIG. 15 shows attachment of an alternative drilling guide configuration to a non-modular prosthesis.

### DETAILED DESCRIPTION OF THE INVENTION

As previously described, femoral head resurfacing or partial replacing implant represents a unique alternative to total hip arthroplasty, particularly in a young and active patient population, who are expected to outlive a conventional total hip replacement. This procedure is a bone-conserving alternative for such patients, wherein the femoral head and neck are preserved, thereby providing greater options to the revision surgeon, should such revision later become necessary. Further advantages include the ability to utilize less invasive, guided surgical techniques, possibly resulting in less damage to soft tissue, muscle and bone; less blood loss, smaller scars, and reduced hospitalization and rehabilitation. Unfortunately heretofore, various factors, including but not limited to vascular damage, bone notching, implant malposition, and the like, have contributed to an unacceptably high failure rate, predominantly evidenced as femoral neck fracture and loosening.

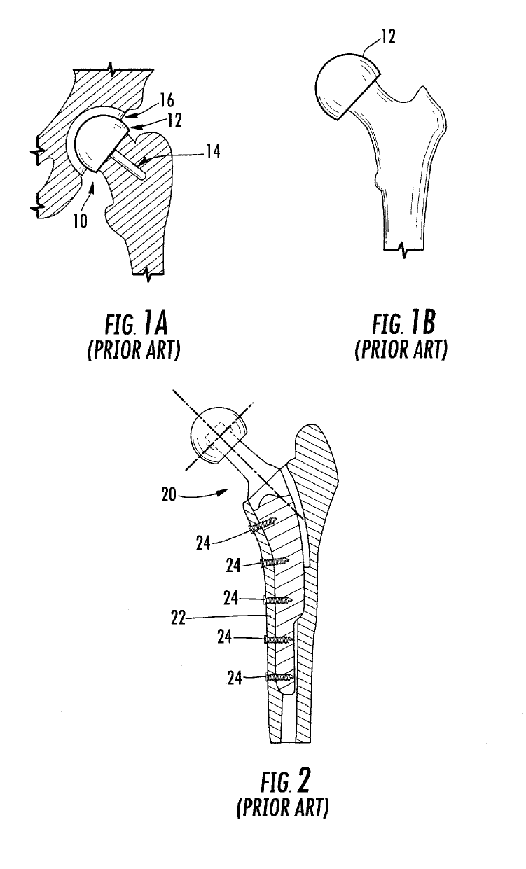

FIGS. 1A and 1B are illustrative of metal-on-metal resurfacing implants currently in use. As illustrated, the prosthesis 10, generally includes a head 12 and a stem 14. as illustrated in FIG. 1A, a bearing surface implant or socket 16 is illustrated. The head 12 and stem 14 components may be of a unitary or modular design. Upon insertion, either cemented or in a cementless configuration, the resurfacing prosthesis of these prior art embodiments are prone to malpositioning, due to difficulty in defining the center of the neck medullary canal, leading to off-center and inaccurate anatomical positioning. This leads to inappropriate bone stress, often causing fracture of the femoral neck or loosening, and possibly excessive wear of the acetabular socket. Such malpositioning often leads to premature failure of the implant, wherein currently the rate of revision surgery for resurfacing procedures is significantly higher that that experienced in total hip arthroplasty, particularly in females.

While the demand for minimally invasive hip resurfacing continues to grow, the aforementioned inherent difficulties limit its use. Patient selection criteria favors use on male patients under the age of 55 who have good bone stock. Use of the technique on obese or female patients is many times problematic, especially in postmenopausal women, where an Australian study indicated a 1.9 fold higher femoral neck fracture rate.

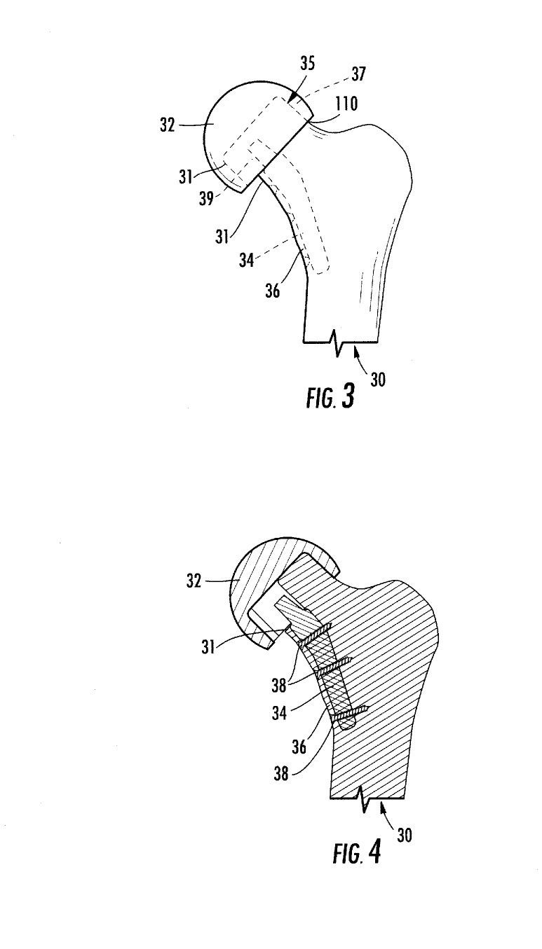

FIG. 2 is illustrative of the prior art total hip prosthesis taught by Tepic et al (U.S. Pat. No. 6,409,768) the contents of which are herein incorporated by reference. Tepic et al teach a total hip arthroplasty 20 which utilizes fixation to the medial cortex 22, achieved through the use of screws 24. The internal plate system has been used successfully for more than 10 years in canine hips (now capturing 80% of the world market). During the last year this technology has also been applied to human total hip replacement with good results. This success has been partly due to the surgical technique which is simple and consistent with previous training of most orthopedic surgeons who commonly use fracture plates and locking nails. This technique allows the surgeon to use MIS exposure but feel confident in the accurate positioning and reproducible secure screw fixation.

The prior art, as represented by the '768 patent, fails to teach or suggest either a unitary or modular design useful for femoral resurfacing, wherein a bone-conserving procedure is provided for partial head replacement while providing a prosthesis which achieves absolute fixation to the medial femoral calcar. In addition, the load sharing concept of the instant invention, provides an internal plate that is sufficiently flexible so as to function as a dynamic loading device, compressing into the hard neck calcar bone, thereby providing better bone interface dynamics, and instigating bone growth. This is extremely important in enhancing the longevity of this treatment.

The present invention provides a unique solution to the problems plaguing the prior art. As illustrated in the following figures, both a modular and unitary design are provided which result in absolute fixation of the implant to cortical bone, thereby eliminating the primary sources of failure, ie neck fracture and implant loosening, and broadening the patient population for whom the procedure is indicated.

Now with reference to FIG. 3, a side perspective view of a modular femoral head resurfacing implant of the invention, generally denoted as 30, is shown wherein the diseased femoral bone has been machined to create a mounting surface 31 upon which a femoral head resurfacing implant 32 is positioned in locking engagement, inclusive of an internal plating component 34 affixed to the medial femoral calcar 36. The head component 32 (either partial or full) will allow for a variety of femoral neck segments to either act as a hemi-arthroplasty (removal of the surface of the femur head) or a total resurfacing component (removal of both the surface of femur head as well as the surface of the acetabulum). Resurfacing implant 32 can be comprised of any number of biocompatible materials, such as but not limited to titanium, cobalt chrome, stainless steel, ceramics or any other material that can serve as a bearing surface. The head component 32 can articulate either on the natural acetabulum or on an acetabular component (not shown) such as one made of cobalt chrome or any other suitable biocompatible material in order to provide for a metal-metal articulation. Although shown as being substantially hemispherical in shape, the present invention contemplates modifications to the shape shown. For example, the head component 32 can be either a full, greater than full, or partial hemisphere. The head component 32 preferably includes a substantially hemispherical outer articulating surface 35 and an inner bearing surface 37 which is intended to bear or abut against the resected surface of the femur head 31 in locking engagement. Such locking engagement may utilize a taper (either self-locking or non-self-locking), wherein in a most preferred embodiment a locking or self-holding taper such as a morse type taper is provided, however it should be noted that other self-holding tapers well known in the art such as the American National, Jacobs, Jarno, Brown and Sharp, British Standard and suitable combinations thereof may be utilized without departing from the scope of the invention.

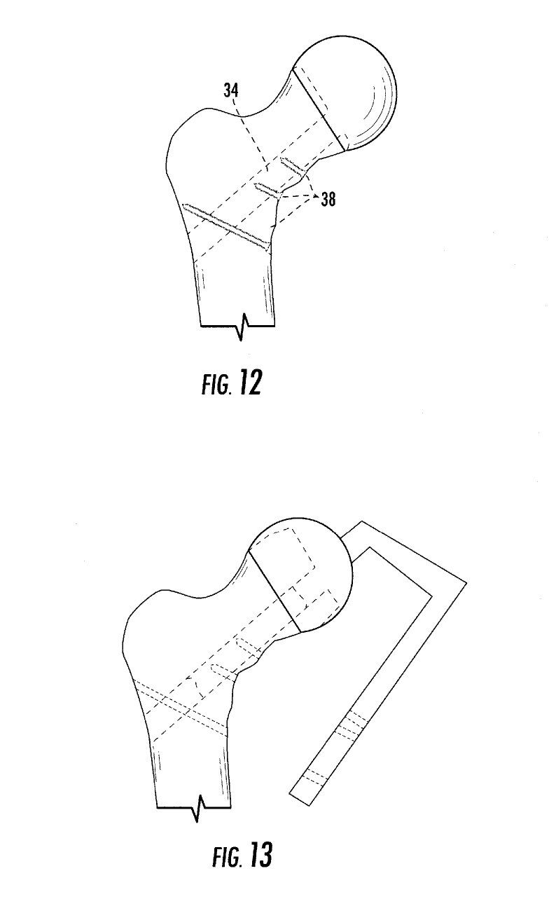

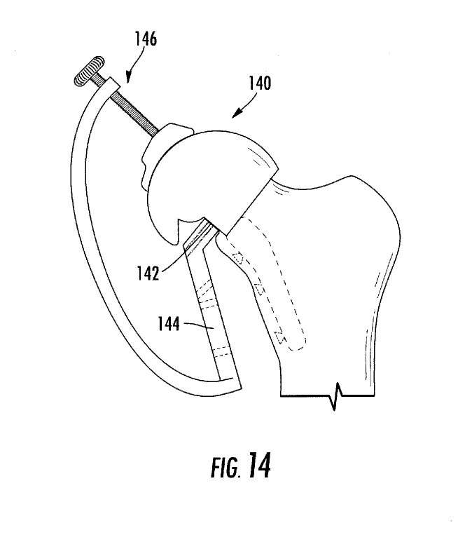

To start the femoral technique, a small curved guide or broach (not shown) is started from the foveal ligament insertion on the femoral head and guided along the internal medial calcar curve 35. This guide, once positioned, allows insertion of the internal plate 34 which is firmly secured to this strong femoral calcar bone 36. This internal plate 34 then provides the secure and accurate positioning of the femoral head reamer (not shown) easily guiding head preparation and adjustable implant positioning, without the need of fluoroscopy. The internal plate 34, in turn is positioned in locking engagement with head component 32, by engagement with a second taper (either self-locking or non-self-locking) formed between the external proximal tip portion 39 of plate 34, with the underside of head 32 within tapered recess 110 as further illustrated in FIG. 11. In alternative contemplated embodiments, the head 32 and internal plate 34 may be of a unitary integral construction, or may be pre-assembled, e.g. via locking tapers or via threaded configurations, which may also be used. The internal plate or stem component 34 can be configured in any number of shapes (e.g., curved as illustrated, or straight (see FIG. 12) or the like suitable shapes. The surface finish of the stem component 34 can be smooth, or utilize a plasma spray, porous coating, polished, grit blasted, or the like, as may be desirable, without departing from the spirit or scope of the invention. The material comprising the stem component 34 can be any biocompatible material such as but not limited to titanium, cobalt chrome, stainless steel, ceramics, and so forth. Although both unitary and modular assemblies are contemplated by the invention, modularity of the head 32 and stem components 34 will allow a surgeon to select from a variety of stem components to match the femoral canal and indications of the particular component. The stem component can be matched with a full or partial head component and provide for full or partial coverage.

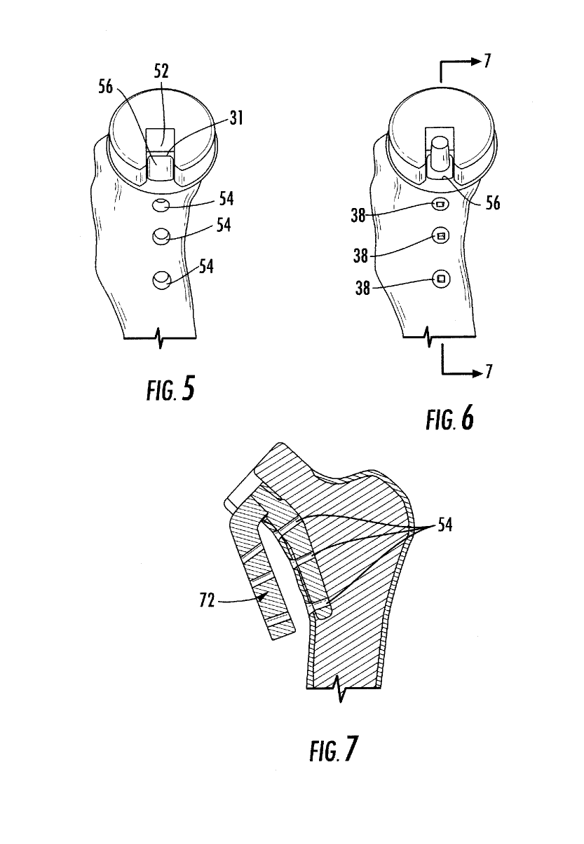

Referring now to FIG. 4, a cross-sectional view of the modular femoral head resurfacing implant 30, is shown. The resurfacing femoral head 32 is illustrated in anti-rotational engagement with both the resurfaced femoral head surface 31 and the internal plating component 34 via tapered connections, and the stem 34 is in turn affixed, via screw fixation 38 (such as the screw technology disclosed by Tepic '768, to the medial femoral calcar 36. In this embodiment, the anti-rotational engagement is derived from formation of a recessed area 52 (see FIG. 5) during machining of the femoral bone surface 31, which provides for mating with member 112 as illustrated in FIG. 11. This illustrative system for anti-rotation is non-limiting, as the invention contemplates alternative means for achieving equivalent functionality, such as the provision of a simpler, albeit functionally equivalent key and keyway designs, machined into the cooperative surfaces of the tapered connections, in similar fashion so as to prevent rotation of the locking elements. Providing the resurfaced femoral bone with such a keyway design also will provide a positive landmark for positioning of a drill guide and head reamers (not shown), particularly in unitary configurations, where initial positioning of the stem 34 is not accomplished.

FIG. 5 further illustrates preparation of the femoral head to fabricate tapered mounting surface 31 having recessed area 52. In this view stem 34 is removed for clarity; however the holes 54 drilled through the medial calcar are illustrated, as is the insertion point 56 for stem 34.

Referring now to FIG. 6, the insertion and screw fixation of the internal plating component or stem 34 of the modular embodiment of FIG. 3 is herein illustrated. Screws 38 are inserted through holes 54 and threaded into preformed holes 94 (see FIG. 9) in the stem 34.

FIG. 7 illustrates a cross-sectional view along line 6-6 of FIG. 6, which further shows mechanical engagement of the drilling guide 72 and internal plating component 34 for alignment of the holes 54 drilled through the medial femoral calcar.

FIG. 8 further illustrates a cross-sectional view illustrating preparation of the femoral head and neck portions in preparation for accepting the prosthesis components.

FIG. 9 shows one embodiment of an internal plate or stem 34 for insertion within the prepared femoral head and neck of FIG. 8. Stem 34 is illustrated as being shaped to permit close affixation to the medial calcar, as well as including preformed holes 94, for accepting screws 38 therein.

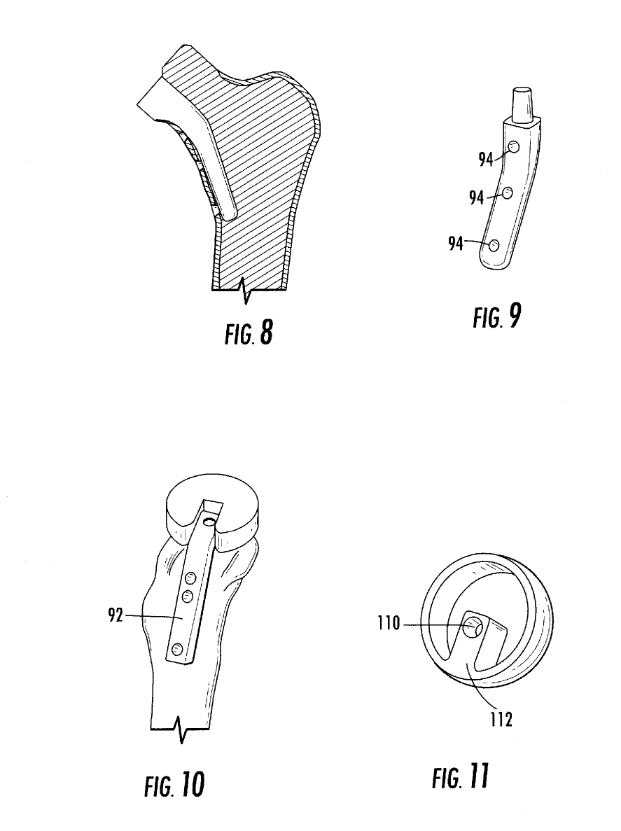

FIG. 10 illustrates a perspective view of the resurfaced femoral head of FIG. 7, into which the internal plating system has been positioned and affixed, and is shown engaged with the drilling guide 72, thereby providing alignment for positioning of holes 54 (not shown).

FIG. 11 shows a prosthetic femoral head 32 of the present invention a illustrating a bone compartment 114, anti-rotation wall 112 and tapered recess 110. The anti-rotation wall 110 acts as a key for insertion within the cut-out 52, which provides a corresponding keyway, to prevent spinning of the components relative to one another. Alternative key/keyway embodiments are contemplated by the invention.

FIG. 12 shows an alternative configuration for an anatomically compliant internal plating component, useful in either a modular or a unitary femoral resurfacing or hemihead implant design. The plating element 34 is a straight component which may have appropriate pre-drilled holes for accepting a variety of screws 38. The fixation can be along the medial calcar as previously described, or alternatively via a screw which crosses through the femur and provides multiple points of fixation, as illustrated. The stem may be of a fixed length in a unitary design, or may be comprised of one or more elements which can be assembled to more closely approximate the anatomical requirements, while conserving inventory requirements.

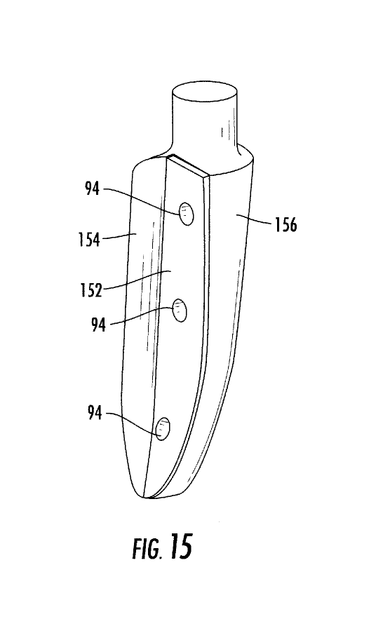

FIG. 13 shows a resurfacing prosthesis in accordance with FIG. 12, illustrating mechanical cooperation with a drill guide;

FIG. 14 illustrates an alternative embodiment wherein a non-modular implant 140 has been inserted. The stem is pre-formed with a tapped hole 142 for fixation of the drilling guide 144, which guide is temporarily further affixed to the top of the implant via a clamping device 146.

FIG. 15 illustrates an alternative internal plating stem 150, which has a rear wall 152, a left wall 154 and a right wall 156. This configuration produces a greater degree of flexibility, which provides a dynamic loading upon fixation to the bone. The dynamic loading functions to transfer weight born by the prosthesis along the medial calcar in a manner which more closely mimics natural transferance of forces in a normal hip. This dynamic flexion of the internal plating system and calcar function to stimulate bone growth and avoid thinning of the medical calcar, often forestalling the need for a total arthroplasty.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A bone-preserving femoral head resurfacing arthroplasty assembly comprising:
a femoral resurfacing implant having a proximal side including a generally hemispherical articulating surface and a distal side having a bearing surface constructed and arranged for substantial abutment against a resected surface of a femoral head; and
an internal plating component having a proximal end and a distal end, extending distally from said distal bearing surface of said femoral resurfacing implant, said internal plating component being constructed and arranged for positive fixation to a medial femoral calcar region.

2. The bone-preserving femoral head resurfacing arthroplasty assembly of claim 1 wherein said assembly is unitary.

3. The bone-preserving femoral head resurfacing arthroplasty assembly of claim 1 wherein said assembly is modular.

4. The bone-preserving femoral head resurfacing arthroplasty assembly of claim 1 wherein said distal side of said femoral resurfacing implant includes at least one tapered recess for receipt of a mating tapered region formed from said resected surface of said femoral head.

5. The bone-preserving femoral head resurfacing arthroplasty assembly of claim 4, wherein said assembly is modular and said distal side of said femoral resurfacing implant further includes at least one tapered recess for receipt of a mating tapered region formed from a proximal tip of said internal plating component.

6. The bone-preserving femoral head resurfacing arthroplasty assembly of claim 4 wherein said taper is a self-holding taper selected from the group consisting of a morse type taper, American National type taper, Jacobs type taper, Jarno type taper, Brown and Sharp type taper, British Standard type taper and suitable combinations thereof.

7. The bone-preserving femoral head resurfacing arthroplasty assembly of claim 5 wherein said taper is a self-holding taper selected from the group consisting of a morse type taper, American National type taper, Jacobs type taper, Jarno type taper, Brown and Sharp type taper, British Standard type taper and suitable combinations thereof.

8. The bone-preserving femoral head resurfacing arthroplasty assembly of claim 1 wherein said distal side bearing surface is formed with a keyway for engagement with said resected surface of said femoral head;
whereby relative rotation therebetween is prevented.

9. The bone-preserving femoral head resurfacing arthroplasty assembly of claim 1 further including a drilling guide constructed and arrange to enable formation of at least one hole in said medial femoral calcar region which is in substantial alignment with mating holes in said distal portion of said internal plating component.

10. The bone-preserving femoral head resurfacing arthroplasty assembly of claim 9 wherein said femoral head resurfacing arthroplasty assembly is modular and said drill guide is constructed and arranged for engagement with said proximal tip of said internal plating element.

11. The bone-preserving femoral head resurfacing arthroplasty assembly of claim 1 wherein said femoral resurfacing implant is formed of a biocompatible material selected from the group consisting of titanium or an alloy thereof, cobalt chrome or an alloy thereof, stainless steel or an alloy thereof, ceramics and combinations thereof.

12. The bone-preserving femoral head resurfacing arthroplasty assembly of claim 1 wherein said femoral resurfacing implant is coated with a plasma spray, hydroxy apatite, a porous coating, and combinations thereof.

13. The bone-preserving femoral head resurfacing arthroplasty assembly of claim 1 wherein said internal plating element is formed of a biocompatible material selected from the group consisting of titanium or an alloy thereof, cobalt chrome or an alloy thereof, stainless steel or an alloy thereof, ceramics and combinations thereof.

14. The bone-preserving femoral head resurfacing arthroplasty assembly of claim 1 wherein said internal plating element is smooth, or utilizes a plasma spray, a porous coating, hydroxy apatite, or is grit blasted.

15. The bone-preserving femoral head resurfacing arthroplasty assembly of claim 1 wherein said internal plating element is constructed and arranged to cooperate with the medial calcar, upon affixation thereto, to provide optimal flexibility effective to provide dynamic compression loading of the femoral neck as drawn into the concave calcar bone of the femoral neck medullary canal.
